(19) United States
(12) Patent Application Publication
(10) Pub. No.: US 2019/0059717 A1
(43) Pub. Date: Feb. 28, 2019

(54) OPTICAL AMPLIFIER, OPTICAL COHERENCE TOMOGRAPHY INCLUDING OPTICAL AMPLIFIER, AND OPTICAL AMPLIFICATION METHOD USING OPTICAL AMPLIFIER

(71) Applicant: CANON KABUSHIKI KAISHA, Tokyo (JP)

(72) Inventor: Takeshi Yoshioka, Ebina-shi (JP)

(21) Appl. No.: 16/170,612

(22) Filed: Oct. 25, 2018

Related U.S. Application Data
(63) Continuation of application No. PCT/JP2017/016689, filed on Apr. 27, 2017.

(30) Foreign Application Priority Data
Apr. 28, 2016 (JP) 2016-091615

(51) Int. Cl. A61B 3/10; A61B 3/12; G01B 9/02; H01S 5/042; H01S 5/026; H01S 5/22; H01S 5/068; H01S 5/50

(52) U.S. Cl. CPC A61B 3/102; A61B 3/1225; G01B 9/02091; G01B 9/02001; H01S 5/3408; H01S 5/026; H01S 5/22; H01S 5/068; H01S 5/50; H01S 5/0425

(57) ABSTRACT

An optical amplifier includes a laminated body including two electrode layers and an active layer disposed therebetween. The laminated body includes a waveguide which guides light in an in-plane direction of the active layer. The light which is incident on the laminated body is amplified and emitted from an end surface in the in-plane direction through the waveguide. At least one of the two electrode layers has an electrode group including at least two electrodes which are disposed separately from each other in a waveguide direction of the waveguide. An amplification factor of the incident light is changeable in accordance with a wavelength of the incident light by independently supplying current to different regions in the active layer using the at least two electrodes. Accordingly, the ASE light including light having an unrequired wavelength may be reduced while sufficient light output intensity is obtained in a required wavelength.

### CROSS-REFERENCE TO RELATED APPLICATIONS

This application is a Continuation of International Patent Application No. PCT/JP2017/016689, filed Apr. 27, 2017, which claims the benefit of Japanese Patent Application No. 2016-091615, filed Apr. 28, 2016, both of which are hereby incorporated by reference herein in their entirety.

### TECHNICAL FIELD

The present invention relates to an optical amplifier which amplifies light output from a wavelength variable light source, an optical coherence tomography including the optical amplifier, and an optical amplification method using the optical amplifier.

### BACKGROUND ART

An optical coherence tomography (OCT) has been widely used as an imaging apparatus for imaging an ocular fundus. In particular, a swept source OCT (hereinafter referred to as “SS-OCT” where appropriate) using a wavelength variable light source has attracted attention. The SS-OCT divides light emitted from the wavelength variable light source into irradiation light which is emitted from the wavelength variable light source and which is incident on an object and reference light and causes the reference light and reflection light which returns from a different depth of the object to interfere with each other. Then the SS-OCT analyzes a frequency component included in a time waveform (an interfering signal) of intensity of the interfering light so as to obtain information on tomography of the object, that is, a tomographic image. The OCT is used in ophthalmology, cardiology, dermatology, and industrial uses including inspection of a semiconductor chip, for example.

Examples of the wavelength variable light source include a wavelength variable light source which changes an oscillation wavelength by deviating one of two reflection mirrors included in a vertical cavity surface emitting laser (VCSEL). As a mechanism for moving a mirror, a mechanism using a microelectromechanical system (MEMS) has been widely used. Hereinafter, such a wavelength variable light source is referred to as “MEMS-VCSEL” where appropriate. MEMS-VCSEL is capable of performing high-speed wavelength variable and obtains a long coherence length. Therefore, the MEMS-VCSEL is suitable for the wavelength variable light source included in the SS-OCT.

Here, the light source used in the OCT preferably outputs light having required intensity so as to obtain an OCT signal having a sufficient S/N ratio. However, if a VCSEL is solely used as the wavelength variable light source, it is difficult to output light of required intensity. Therefore, according to NPL1, light emitted from the MEMS-VCSEL is subjected to induction amplification using a booster optical amplifier (BOA) so that required light output intensity is obtained.

### CITATION LIST

### Non Patent Literature

NPL 1 Journal of Lightwave Technology 33(16) p. 3461-3468

Here, the inventor finds out a problem to be solved in amplification of light output intensity using the BOA disclosed in NPL1. Specifically, amplified spontaneous emission (ASE) light is generated from the BOA when light is amplified using the BOA. The ASE light is spontaneous emitted light generated from the BOA, and the ASE light includes light of wavelengths other than a wavelength to be amplified. Therefore, an OCT signal obtained when light including the ASE light is emitted includes noise.

NPL1 discloses a temporal change of an amplification factor of the BOA relative to incident light having a wavelength which is temporally changed. The amplification factor is increased by increasing an amount of current to be supplied to the BOA so that light having intensity required in a certain wavelength may be obtained. However, even if light output intensity required in a certain wavelength is obtained only by controlling a current amount, intensity of ASE light including unrequired wavelengths may be increased. NPL1 does not discloses control for reducing such ASE light of the BOA.

Accordingly, the present invention provides an optical amplifier capable of reducing ASE light including light having unrequired wavelengths while sufficient light output intensity is obtained in a required wavelength.

### SUMMARY OF INVENTION

According to an embodiment of the present disclosure, an optical amplifier includes a laminated body including two electrode layers and an active layer disposed between the electrode layers. The laminated body includes a waveguide which guides light in an in-plane direction of the active layer. The light which is incident on the laminated body is amplified and emitted from an end surface in the in-plane direction of the laminated body through the waveguide. At least one of the two electrode layers has an electrode group including at least two electrodes which are disposed separately from each other in a waveguide direction of the waveguide. An amplification factor of the incident light is changeable in accordance with a wavelength of the incident light by independently supplying current to different regions in the active layer using the at least two electrodes.

Further features of the present invention will become apparent from the following description of exemplary embodiments with reference to the attached drawings.

### BRIEF DESCRIPTION OF DRAWINGS

FIG. 1 is a diagram illustrating an example of a configuration of a wavelength sweeping light source and a configuration of a semiconductor optical amplifier (SOA) according to a first embodiment of the present invention.

FIG. 2A is a perspective view of the SOA and FIG. 2B is a top view of the SOA according to the first embodiment of the present invention.

FIG. 3A is a top view of an electrode region (taken along a line IIIA to IIIA of FIG. 2B) of the SOA, FIG. 3B is a cross-sectional view of a non-electrode region (taken along a line IIIB to IIIB of FIG. 2B), and FIG. 3C is a cross-sectional view of an optical waveguide (taken along a line IIIC to IIIC of FIG. 2B) according to the first embodiment of the present invention.

FIG. 4 is a graph illustrating a sweeping spectrum of MEMS-VCSEL in 1060 nm band.

FIG. 5 is a graph illustrating a sweeping spectrum of emitted light which is a target in the SOA according to the first embodiment of the present invention.

FIG. 6 is a graph illustrating a gain spectrum in an active layer of the SOA according to the first embodiment of the present invention.

FIG. 7 is a graph illustrating the relationship between a carrier density N in the SOA according to the first embodiment of the present invention and a sum ∫g(N) of positive gains obtained in a target wavelength range.

FIG. 8 is a graph illustrating the relationship between the carrier density N in the SOA according to the first embodiment of the present invention and an obtained gain g (N, λ=1040).

FIG. 9 is a graph illustrating the relationship between the carrier density N in the SOA according to the first embodiment of the present invention and g (N, λ=1040)/∫g(N).

FIG. 10 is a graph illustrating the relationship between an incident light wavelength λ and L_{g}, N_{g}, and Nₐ for an optimum driving state in the SOA according to the first embodiment of the present invention.

FIG. 11 is a graph illustrating the relationship between an incident light wavelength λ and the carrier densities N in various electrode regions for the optimum driving state in the SOA according to the first embodiment of the present invention.

FIG. 12A is a graph illustrating the relationships between the wavelength λ and g(N, γ)·L which attains an optimum driving state in an incident light wavelength of 1030 nm in the SOA of the first embodiment of the present invention and an SOA of a single electrode structure, and FIG. 12B is a graph illustrating the relationships between the wavelength λ and g(N, γ)·L which attains an optimum driving state in an incident light wavelength of 1060 nm in the SOA of the first embodiment of the present invention and the SOA of a single electrode structure.

FIG. 13 is a plane view of an SOA according to a second embodiment.

FIG. 14 is a graph illustrating the relationship between an incident light wavelength λ and carrier densities N in various electrode regions for attaining an optimum driving state in the SOA according to the second embodiment of the present invention.

FIG. 15A is a graph illustrating the relationships between a wavelength λ and g(N, λ)·L which attains an optimum driving state in an incident light wavelength of 1030 nm in the SOA of the second embodiment of the present invention and an SOA of a single electrode structure, and FIG. 15B is a graph illustrating the relationship between the wavelength λ and g(N, λ)·L which attains an optimum driving state in an incident light wavelength of 1060 nm in the SOA of the second embodiment of the present invention and the SOA of a single electrode structure.

FIG. 16 is a plane view of an SOA according to a third embodiment.

FIG. 17A is a graph illustrating the relationship between a wavelength λ and g(N, λ)·L which attains an optimum driving state in an incident light wavelength of 1030 nm in an SOA of a third embodiment of the present invention and an SOA of a single electrode structure, and FIG. 17B is a graph illustrating the relationship between the wavelength λ and g(N, λ)·L which attains an optimum driving state in an incident light wavelength of 1060 nm in the SOA of the third embodiment of the present invention and the SOA of a single electrode structure.

FIG. 18 is a graph illustrating a sweeping spectrum of emitted light which is a target in an SOA according to a fourth embodiment of the present invention.

FIG. 19 is a graph illustrating the relationship between an incident light wavelength λ and L_{g}, N_{g}, and Nₐ for an optimum driving state in the SOA according to the fourth embodiment of the present invention.

FIG. 20 is a graph illustrating the relationship between an incident light wavelength λ and carrier densities N in various electrode regions for attaining the optimum driving state in the SOA according to the fourth embodiment of the present invention.

FIG. 21A is a graph illustrating the relationship between the wavelength λ and g(N, λ)·L which attains the optimum driving state in an incident light wavelength of 1030 nm in the SOA of the fourth embodiment of the present invention and an SOA of a single electrode structure, and FIG. 21B is a graph illustrating the relationship between the wavelength λ and g(N, λ)·L which attains the optimum driving state in an incident light wavelength of 1060 nm in the SOA of the fourth embodiment of the present invention and the SOA of a single electrode structure.

FIG. 22 is a diagram illustrating an example of a configuration of an optical coherence tomographic imaging apparatus using an SOA according to a fifth embodiment of the present invention.

FIG. 23 is a table of examples of combinations of electrode lengths which attain an optimum gain length in the SOA according to the third embodiment of the present invention and carrier densities N for attaining an optimum driving state in various electrode regions.

### DESCRIPTION OF EMBODIMENTS

An optical amplifier according to an embodiment of the present invention will be described. However, the present invention is not limited to this.

### Optical Amplifier

An optical amplifier according to this embodiment has a configuration of a laminated body including two electrode layers and an active layer interposed between the electrode layers. As an example of the laminated body, a lower electrode layer, a lower cladding layer, an active layer, an upper cladding layer, a contact layer, and an upper electrode layer are laminated in this order. The laminated body configured by a semiconductor is referred to as a “semiconductor optical amplifier (SOA)”. Light which is incident on the SOA is referred to as “incident light” and light which is emitted from the SOA is referred to as “emitted light” where appropriate hereinafter.

Furthermore, an end surface of the laminated body of the SOA on which light is incident is referred to as an “incoming end surface” and an end surface from which light is emitted is referred to as an “outgoing end surface” where appropriate.

The laminated body has a waveguide where light is wave-guided in an in-plane direction of the active layer. Light which is incident on an end surface of the laminated body in the in-plane direction (on an incoming end surface side) is emitted through the waveguide after being amplified by the other end surface of the laminated body in the in-plane direction (on an outgoing end surface side). As a waveguide structure, the upper electrode layer, the upper contact layer, and the upper cladding layer constitute a ridge structure.

Furthermore, at least one of the two electrode layers disposed on an upper side and a lower side relative to the active layer has an electrode group including at least two electrodes which are disposed separately from each other in a waveguide direction of the waveguide.

The optical amplifier according to this embodiment supplies current to individual regions in the active layer using the at least two electrodes included in the electrode group so as to change an optical amplification factor in accordance with a wavelength of incident light.

Note that the optical amplifier may include a controller which supplies current to the individual regions in the active layer.

### Control of Optical Amplification Factor Based on Wavelength of Incident Light

The optical amplifier according to this embodiment changes an amplification factor of incident light in accordance with a wavelength of the incident light so as to selectively amplify only a required wavelength, obtain sufficient optical output intensity, and suppress generation of ASE light including light of unrequired wavelengths which are other than the required wavelength as much as possible. In a case where light of a wavelength λ₁ in the incident light is to be amplified, an amount of current to be supplied to the optical amplifier is controlled so that light having a wavelength of λ₁ is output from the optical amplifier with sufficient optical output intensity. If ASE light including an unrequired wavelength is included in emitted light, a region of the optical amplifier which amplifies light after current is supplied to the optical amplifier is reduced so that generation of the ASE light including a wavelength other than λ₁ is reduced. Specifically, in addition to an amount of current supplied to the optical amplifier (current density), a region in which light is amplified is changed, a specific wavelength is amplified and amplification of other wavelengths is suppressed. The region in which light is amplified may be controlled since the electrode layers included in the laminated body are separately disposed in accordance with a plurality of electrodes, and therefore, current supply may be independently controlled. The control of an amount of current supplied to the individual electrodes is performed by the controller. The optical amplifier and the controller may be collectively referred to as a “light source system”.

Note that the region in which the light is amplified may be referred to as a gain length.

Here, a region in the waveguide in which a positive gain is obtained in a wavelength of incident light is defined as a gain region, and a total length of gain regions along the waveguide is defined as a gain length. Specifically, as a wavelength of incident light becomes longer, a gain length is set longer so that light of a long wavelength may be selectively amplified. On the other hand, as a wavelength of incident light becomes shorter, a gain length is set shorter so that light of a short wavelength may be selectively amplified. As a method for reducing the gain length, the number of electrodes used for supply of current to the active layer is reduced. Accordingly, by reducing the number of electrodes used for supply of current to the active layer of the optical amplifier as a wavelength is shorter, light of a short wavelength may be selectively amplified. The opposite is equally true.

Furthermore, when a density of current supplied to the active layer is high, light of a short wavelength is easily amplified and light of a long wavelength is difficult to be amplified. Therefore, as a wavelength of incident light is shorter, a density of current supplied to the active layer is preferably increased.

Note that it is preferable that a waveform (a spectrum form) of a temporal change of a wavelength of light emitted from the optical amplifier is a substantially Gaussian form or a substantially cosine taper form. This is because an OCT image having less noise is easily obtained if light having such a spectrum form is used as OCT measurement light.

Here, the substantially Gaussian form, a substantially rectangle form, and the substantially cosine taper form are concept including forms which are slightly different from a Gaussian form and a cosine taper form as long as large noise is not included in an OCT image.

Furthermore, in a case where the electrode group includes at least three electrodes and current is not supplied to at least one of the electrodes in the electrode group, it is preferable that the electrode which does not receive the current is not one of the electrodes which is disposed closest to the incoming end surface of the laminated body.

Furthermore, it is preferable that one of the electrodes which does not receive supplied current is disposed closest to an end surface from which light is emitted.

Furthermore, it is preferable that current densities in the electrode group are substantially equal to each other.

A gain in a single optical amplifier may be temporally changed mainly by current density in an electrode region. In regions on the optical waveguide, a region in which a positive gain is obtained at a center wavelength of incident light is determined as a gain region whereas a region in which the gain is equal to or smaller than zero is determined as a non-gain region.

Hereinafter, the optical amplifier according to the embodiment of the present invention will be described in detail using a concrete configuration. A configuration, a size, material, and a control method described in embodiments below are merely examples, and the present invention is not limited to these.

Note that, hereinafter, a configuration including an SOA serving as an optical amplifier and a wavelength sweeping light source which sweeps a wavelength of emitted light in an MEMS mechanism serving as a light source will be described as an example.

Furthermore, in the description below, MEMS-VCSEL described above is taken as an example of the wavelength sweeping light source which performs wavelength sweeping in the MEMS mechanism. MEMS-VCSEL of this embodiment displaces one of mirrors included in a resonator of VCSEL (which is referred to as an MEMS mirror where appropriate) by electrostatic attractive force.

### First Embodiment

A first embodiment of the present invention will be described hereinafter.

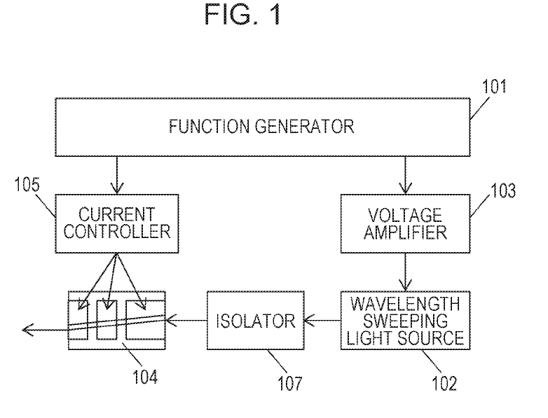

An SOA and a wavelength sweeping light source according to this embodiment will be described with reference to FIG. 1.

First, a function generator 101 transmits the same signal to a voltage amplifier 103 which controls MEMS driving of a wavelength sweeping light source 102 and a current control device (controller) 105 which controls driving of a SOA 104. By this, the MEMS driving of the wavelength sweeping light source 102 and the driving of the SOA 104 may be temporally synchronized with each other. Accordingly, by recognizing the relationship between a voltage value for controlling displacement of the MEMS mirror of the wavelength sweeping light source 102 and an oscillation wavelength in advance, a driving current value of the SOA 104 may be controlled in accordance with the oscillation wavelength.

Furthermore, an isolator 107 is disposed between the wavelength sweeping light source 102 and the SOA 104 so as to suppress light which returns to the wavelength sweeping light source 102.

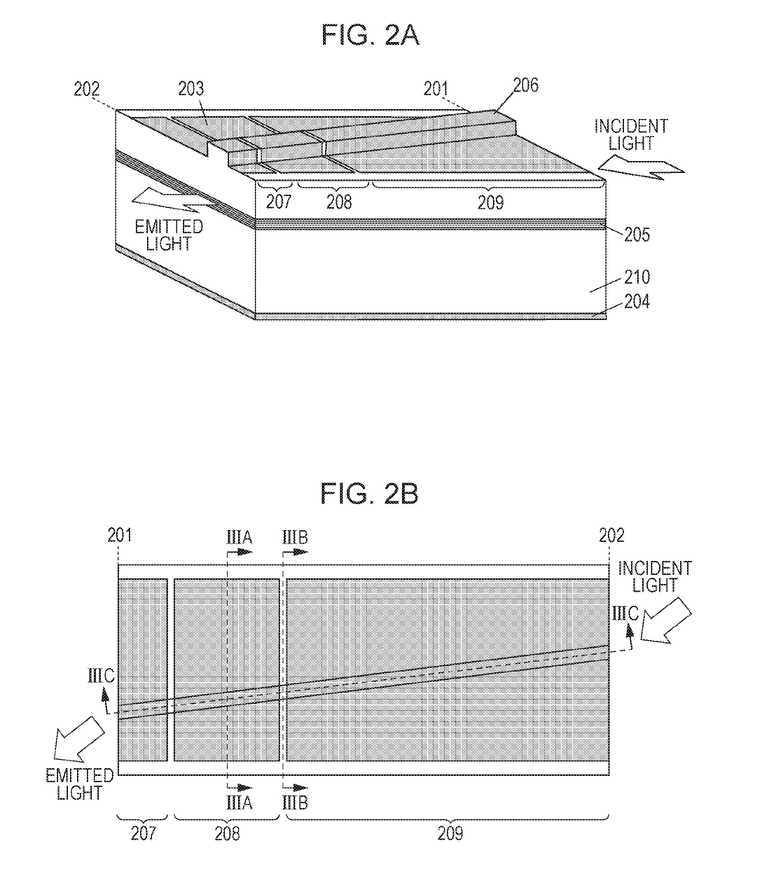

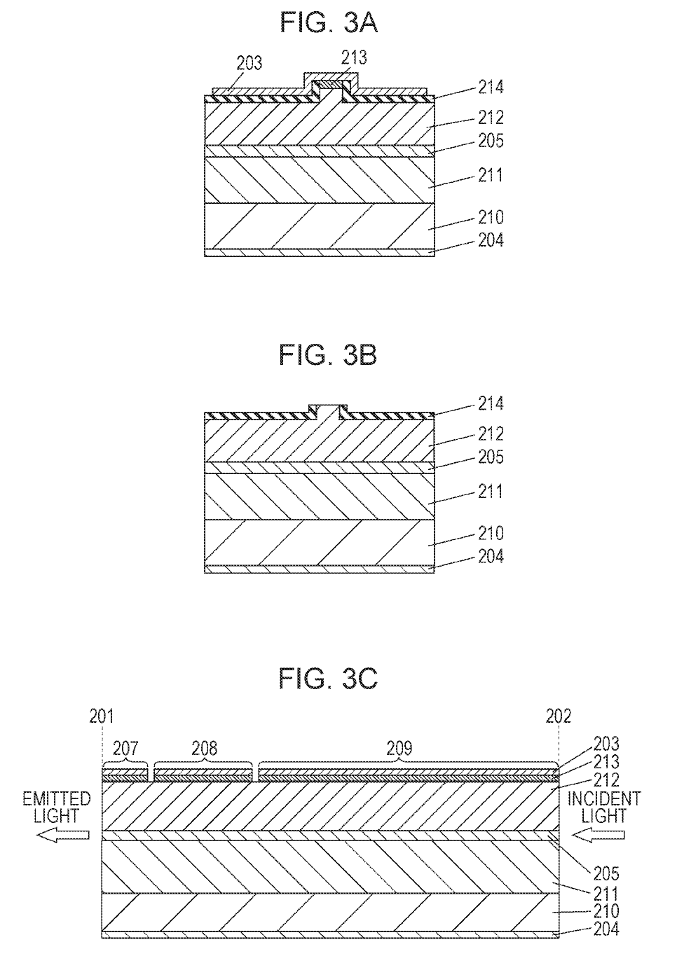

Next, a configuration of the SOA 104 according to this embodiment will be described with reference to FIGS. 2A and 2B and FIGS. 3A to 3C.

FIG. 2A is a perspective view of the SOA 104 according to this embodiment, and FIG. 2B is a top view of the SOA 104. FIG. 3A is a cross-sectional view of a region including the electrodes of the SOA 104 according to this embodiment disposed therein (a cross section taken along a line IIIA to IIIA of FIG. 2B), and FIG. 3B is a cross-sectional view of a non-electrode region (a cross section taken along a line IIIB to IIIB of FIG. 2B) which does not include electrodes. FIG. 3C is a cross-sectional view of the optical waveguide of the SOA 104 (a cross section taken along a line IIIC to IIIC of FIG. 2B) according to this embodiment.

Note that, the SOA 104 has a mechanism in which all the electrodes are connected to a driving system (a driver) as illustrated in FIG. 1 and amounts of current to be supplied to the active layer (current density) may be individually controlled for individual electrode regions.

Next, a procedure of fabrication of the SOA 104 according to this embodiment will be described.

First, n-Al_{0.9}GaAs is successively subjected to epitaxial growth so as to form an n-type cladding layer 211 on a GaAs substrate 210 by a metal organic chemical vapor deposition (MOCVD) method, for example. Similarly, GaIn_{0.3}As of a single quantum well structure, p-Al_{0.9}GaAs, and highly doped p-GaAs are successively subjected to the epitaxial growth so as to form an active layer 205, a p-type cladding layer 212, and a contact layer 213, respectively. A wafer formed by laminating the layers is processed by a general photolithography method and wet/dry etching so that a ridge 206 is formed and an optical waveguide is thus formed. Since the ridge 206 is formed, light may be shielded in a portion of the waveguide in the active layer and waves may be guided. For example, after SiO₂ is formed by a spattering method, a stripe mask is formed by a photolithography method using a photoresist to form an optical waveguide. Thereafter, the semiconductor other than a portion corresponding to SiO₂ is selectively removed by wet etching and the semiconductor other than the mask is selectively removed by dry etching. Here, the portions are removed until certain portions in the contact layer 213 and the p-type cladding layer 212 are reached. The optical waveguide has a width of 3 μm so that a single mode is attained. The optical waveguide is inclined by approximately 7 degrees relative to normal directions of an incoming end surface 201 and an outgoing end surface 202 near the end surfaces 201 and 202 so that reflection in the incoming end surface 201 and the outgoing end surface 202 is suppressed.

Next, a p-electrode 203 is formed by a vacuum deposition method and photolithography. The p-electrode 203 is formed by Ti/Au, for example, and a plurality of p-electrodes 203 are arranged on the optical waveguide in series relative to a waveguide direction in a state in which the individual p-electrodes 203 are insulated. Furthermore, the contact layer 213 of the non-electrode region is removed by wet etching using citric acid so as to be a region electrically isolated.

Before an n-electrode 204 is formed, a thickness of the substrate 210 is reduced to approximately 100 μm by polishing. By this, cleavage on a facet surface is facilitated. Subsequently, the n-electrode 204 is formed by a vacuum deposition method. The n-electrode 204 is formed of AuGe/Ni/Au, for example. To obtain preferred electric characteristics, annealing is performed in a high-temperature nitrogen atmosphere so that the electrodes and the semiconductors become alloy. Finally, facet surfaces are formed on the incoming end surface 201 and the outgoing end surface 202 by the cleavage so that an element of the SOA 104 is formed.

The forming method, the semiconductor material, the electrode material, and the dielectric material are not limited to those disclosed in the embodiment, and other methods and material may be used without departing from the scope of the present disclosure. For example, a p-type GaAs substrate may be used as the substrate 210, and in this case, conductive types of the individual semiconductor layers are appropriately changed.

Although the active layer 205 has the single quantum well (SQW) structure in the example, the active layer 205 may have a multiquantum well (MQW) structure having a plurality of quantum wells. As the MQW structure, the quantum wells may have the same composition and the same well width, or an asymmetry multiquantum well (A-MQW) structure (an asymmetry quantum well structure) in which at least one of a plurality of quantum wells has a different composition or a different well width may be employed.

Furthermore, material of the quantum well is also not limited to the foregoing examples, and light emitting material, such as GaAs, GaInP, AlGaInN, AlGaInAsP, or AlGaAsSb, may be used.

Although the active layer 205 has a uniform thickness and single composition in a waveguide direction, the present invention is not limited to this as long as the effects of the invention are obtained.

Although the optical waveguide has a straight shape having a constant width and a constant refractive index, the present invention is not limited to this as long as the effects of the present invention are obtained. For example, the optical waveguide has a curved shape or a branched shape, or may be configured such that a width and a refraction index of the optical waveguide are changed in a waveguide direction.

Furthermore, although the optical waveguide has the width of 3 μm as an example so that a single mode of the light emitted from the SOA is attained, a multi-mode may be attained.

Moreover, although the case where the ridge type optical waveguide is employed as the optical waveguide is described as an example, current or light may be shielded by employing a stripe active layer or a current block layer.

Although the case where the SOA of this embodiment has the optical waveguide which is inclined by approximately 7 degrees relative to the normal directions of the incoming end surface and the outgoing end surface near the end surfaces so as to suppress reflection at the end surfaces is described as an example, the angle is not limited to 7 degrees as long as the effects of the present invention are attained.

Although the case where the three electrodes are included in the electrode group is described as an example in this embodiment, the present invention is not limited to this as long as the number of electrodes satisfies required conditions for obtaining the effects of the present invention (at least two electrodes).

A non-gain region (a window structure) may be formed near the end surfaces so that concentration of light and current at the incoming end surface 201, the outgoing end surface 202, or both of the end surfaces is suppressed.

An anti-reflection (AR) film may be formed on the incoming end surface 201 and the outgoing end surface 202 so that reflection at the incoming end surface 201, the outgoing end surface 202, or both of the end surfaces is suppressed.

Although the case where the plurality of p-electrodes 203 are arranged in series relative to the waveguide direction is described as an example, a plurality of n-electrodes 204 or both of the p-electrodes 203 and the n-electrodes 204 may be arranged.

Although the case where a length of the non-electrode region is constant on the waveguide is described as an example, the present invention is not limited to this as long as the effects of the present invention are attained.

Next, driving states of the plurality of electrodes will be described.

Note that, although a driving state of the SOA is defined by carrier density hereinafter, current densities in individual electrode regions are adjusted so that desired values of the carrier densities in a gain region and non-gain region are obtained in practice.

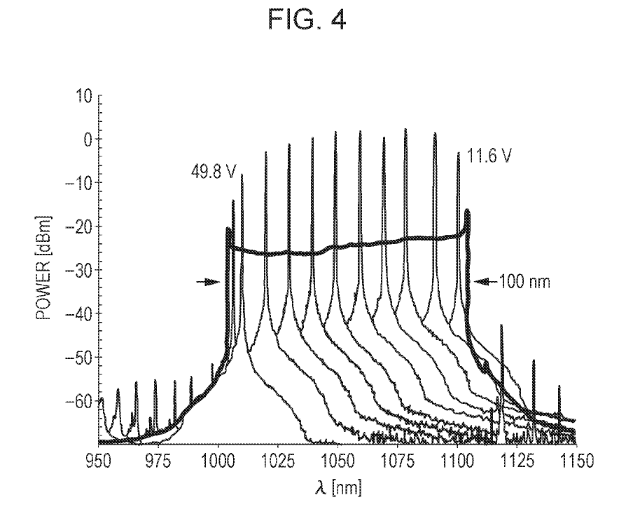

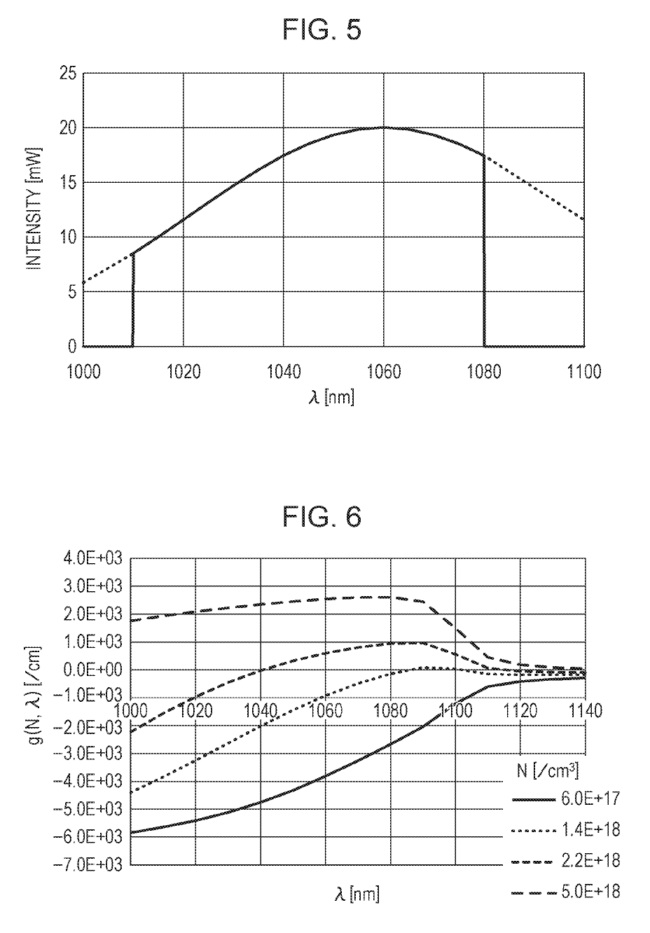

In this embodiment, a MEMS-VCSEL sweeping spectrum having a center wavelength in the vicinity of 1060 nm (FIG. 4) is assumed as incident light and a sweeping spectrum shape represented by Expressions below is assumed as target emitted light (illustrated as a solid line in FIG. 5). Electron Lett 2012 Oct 11 48(21) 1331-1333 is cited in FIG. 4.

$$\begin{matrix}1010 \leq λ [ nm ] \leq 1080 \text{:} \\ P = 20 \cdot exp [ - \frac{{( λ - μ )}^{2}}{2 {σ}^{2}} ] \\ σ = \frac{( FWHM )}{\sqrt{2 ln 2}} = \frac{90}{\sqrt{2 ln 2}} \\ μ = 1060 & ( i ) \\ λ [ nm ] < 1010 , 1080 < λ [ nm ] \text{:} \\ P = 0 & ( ii )\end{matrix}$$

Here, λ denotes a wavelength and P denotes light intensity.

The sweeping spectrum has a Gaussian form (illustrated by a dotted line in FIG. 5) having a center wavelength of 1060 nm, a light intensity in the center wavelength of 20 mW, and a full width at half maximum of 90 nm in a sweeping wavelength range (1010 nm to 1080 nm) of the incident light. The sweeping spectrum has a form having a light intensity of 0 mW in a wavelength range other than the sweeping wavelength range.

Furthermore, the three electrodes are employed as illustrated in FIGS. 2A and 2B according to this embodiment and the gain region and the non-gain region are separated from each other as division of electrode regions, the plurality of gain regions have the same carrier density. Furthermore, carrier density corresponding to a gain of an incident light wavelength of zero is obtained in the non-gain region.

In the SOA 104, a state which satisfies the following expression is required so that a sweeping spectrum of incident light is output as a target sweeping spectrum.

Pₒᵤₜ(λ)=Pᵢₙ(λ)·exp[g(N,+)·L_{g}·Γ] Expression A

It is assumed here that λ denotes a wavelength (1010 nm to 1080 nm), Pᵢₙ(λ) denotes incident light intensity in the wavelength λ, and Pₒᵤₜ(λ) denotes emitted light intensity in the wavelength λ.

Furthermore, it is assumed that g(N, λ) denotes a gain of the SOA 104 in the wavelength λ in a carrier density N, L_{g} denotes a total length of gain regions in the SOA 104, and Γ denotes a confinement factor in the optical waveguide of the SOA 104. Hereinafter, a result of calculation in a case where Γ is 0.03 is illustrated.

A gain spectrum in the active layer of the SOA 104 according to this embodiment is illustrated in FIG. 6.

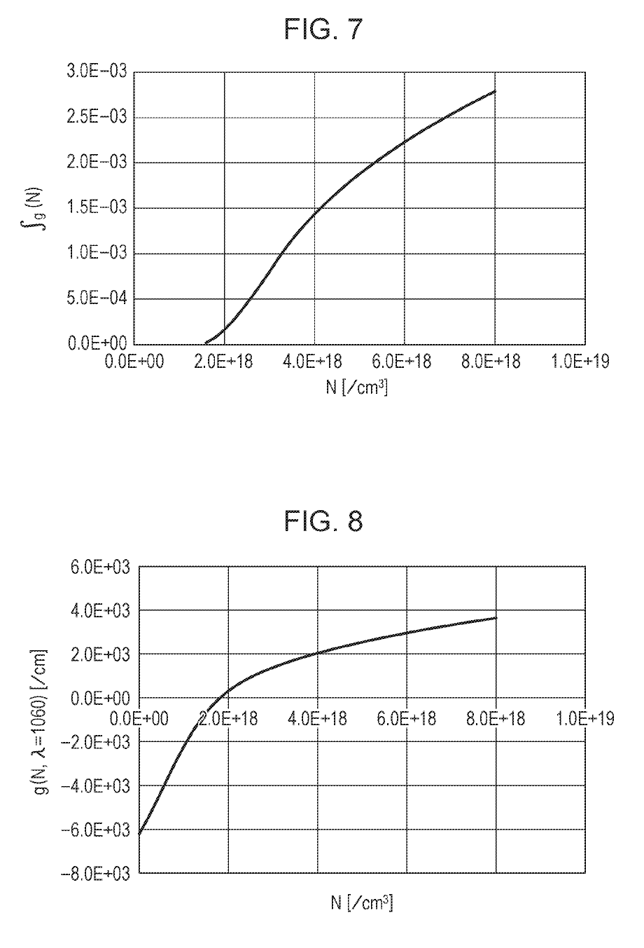

Furthermore, the relationship between the carrier density N and a sum ∫g(N) of positive gains obtained in the target wavelength range is obtained based on the gain spectrum (FIG. 7). ∫g(N) is used as an index representing a total amount of the ASE light per unit length of a gain region generated from the SOA 104 in the carrier density N in the target wavelength range.

For example, a method for deriving an optimum driving state in an incident light wavelength of 1060 nm is illustrated below.

The optimum driving state indicates a driving state in which incident light in a certain wavelength (the wavelength of 1060 nm in this case) is amplified and the ASE light is reduced as much as possible so that a desired sweeping spectrum is formed. The driving state indicates combinations of lengths of the electrode regions and carrier densities in the electrode regions. Specifically, assuming that a total length (a gain length) and carrier density of the gain region are denoted by L_{g} and N_{g}, and a total length (a non-gain length) of a non-gain region and carrier density are denoted by Lₐ and Nₐ, four values for determining the optimum driving state are required to be determined.

First, N_{g} and Nₐ for attaining the optimum driving state are obtained.

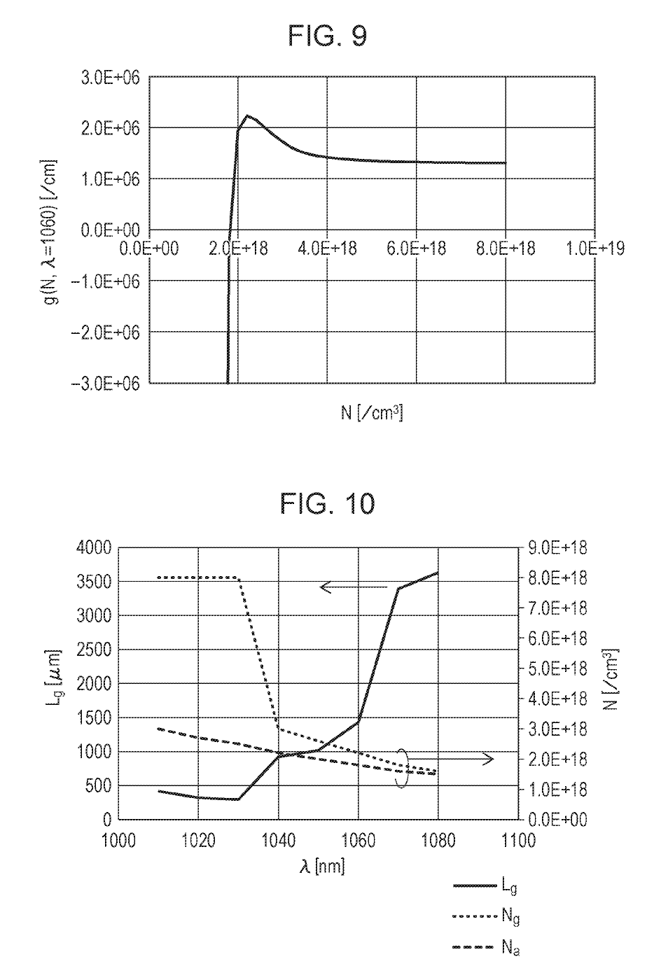

The relationship between the carrier density N and an obtained gain g (N, λ=1060) in the wavelength of 1060 nm is illustrated in FIG. 8. Next, a result of calculation “g(N, λ=1060)/∫g(N)” based on the relationship in FIG. 8 is illustrated in FIG. 9. “g(N, λ)/∫g(N)” represents a rate of gain in the wavelength λ to a total amount of the ASE light in the target wavelength range. As the value is increased, incident light in the wavelength λ may be efficiently amplified while an amount of the ASE light is suppressed. The value of “g(N, λ=1060)/∫g(N)” becomes maximum in FIG. 9 when N is “2.2E+18/cm³”, and therefore, N_{g} which satisfies the optimum driving state in the incident light wavelength of 1060 nm is determined to be “2.2E+18/cm³”. On the other hand, Nₐ which attains the optimum driving state is determined to be “1.8E+18/cm³” which corresponds to “g(N, λ=1060)=0” according to FIG. 8 since Nₐ indicates carrier density which attains a gain of zero in the wavelength 1060 nm.

Next, L_{g} for attaining the optimum driving state is obtained.

This length is obtained by assigning Pᵢₙ (λ=1060) of 1.55 [mW] according to FIG. 4, Pₒᵤₜ (λ=1060) of 20 [mW] according to FIG. 5, and g (N=2.2E+18, λ=1060) of 597 [/cm], and Γ of 0.03 according to FIG. 6 to Expression A. As a result, L_{g} of 1429 [μm] is derived.

Accordingly, the optimum driving state in the incident light wavelength of 1060 nm is determined as follows: N_{g}=2.2E+18 [/cm3], Nₐ=1.8E+18 [/cm3], and L_{g}=1429 [μm].

Similarly, results of obtainment of L_{g}, N_{g}, and Nₐ which attain the optimum driving state in the target wavelength range from 1010 nm to 1080 nm are illustrated in FIG. 10. Basically, as the incident light has a longer wavelength, Lₐ which attains the optimum driving state tends to be longer and N_{g} and Nₐ tend to be lower.

Since the number of electrodes is 3 in this embodiment, optimum gain lengths may be obtained for at least three wavelengths in the incident light. It is assumed that the optimum driving state is to be set such that optimum gain lengths for incident light wavelengths of 1010 nm, 1040 nm, and 1080 nm are obtained, for example. The values of L_{g} for the optimum driving states in the incident light wavelengths of 1010 nm, 1040 nm, and 1080 nm are 417 μm, 920 μm, and 3630 μm, respectively, according to FIG. 10. Accordingly, assuming that a length of an n-th electrode is denoted by Lₙ and L₁, L₂, and L₃ in FIG. 3 are 417 μm, 504 μm, and 2709 μm (in random order), values of L_{g} which attain the optimum driving states relative to the three types of incident light wavelength selected as a combination may be obtained. Then, Lₐ may be determined as follows: Lₐ=L₁+L₂+L₃−L_{g}.

Although gain lengths which attain optimum driving states may not be obtained in wavelengths other than the selected three wavelengths, combinations which are most similar to the combinations of the electrode lengths are considered so that the optimum driving states may be substantially attained.

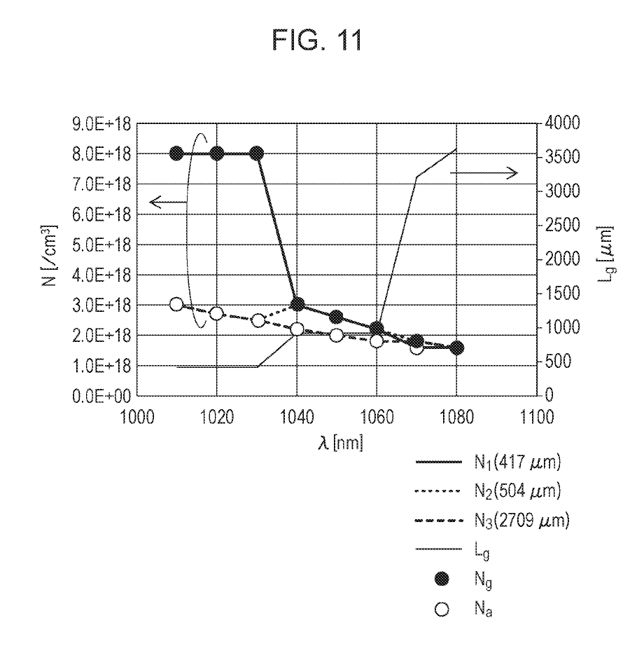

Accordingly, assuming that a carrier density in an n-th electrode region is denoted by Nₙ, N₁, N₂, N₃, and L_{g} which attain the optimum driving state for the incident light wavelength λ are summarized as illustrated in FIG. 11.

Note that plot points of N_{g} are denoted by black circles and plot points of Nₐ are denoted by white circles.

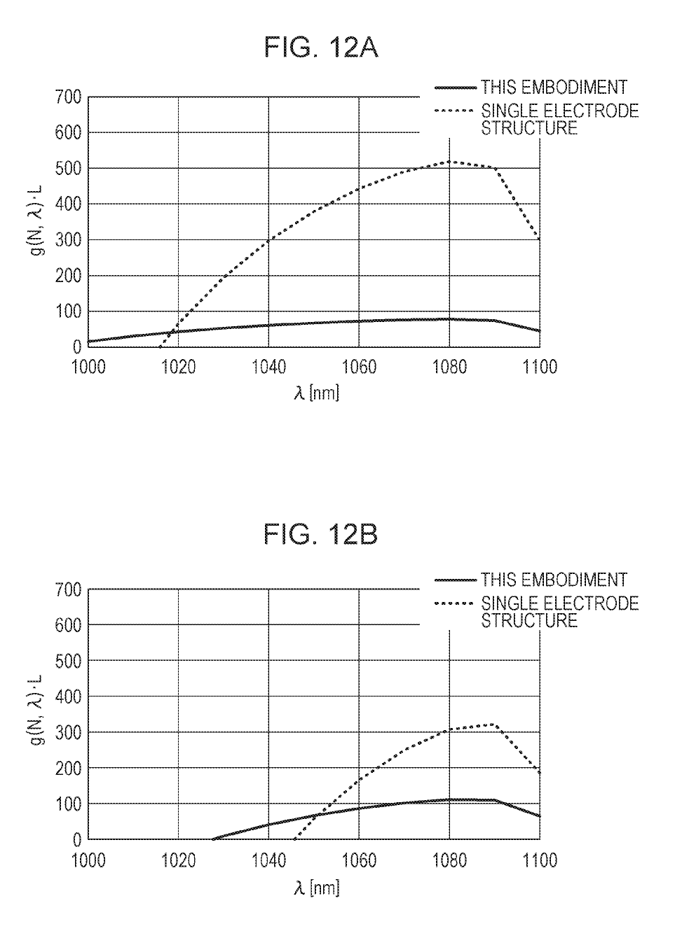

When g(N, λ)·L which attains the optimum driving states in the incident light wavelengths of 1030 nm and 1060 nm in the SOA in the driving state of this embodiment and those in an SOA having a single electrode structure are compared with each other, results in FIGS. 12A and 12B are obtained, respectively. According to the comparison in the same incident light wavelength, an amount of ASE light generated in the SOA in the driving state according to this embodiment may be considerably reduced than that in the SOA having the single electrode structure.

According to this embodiment, the calculation is performed on the assumption that a sweeping spectrum of the Gaussian form having a center wavelength of 1060 nm, a light intensity at a center wavelength of 20 mW, and a full width at half maximum of 90 nm is used as target emitted light. However, in this embodiment, a center wavelength, light intensity at the center wavelength, a full width at half maximum, and a sweeping spectrum form are not limited to these (as for a rectangle form, refer to a fourth embodiment).

Although the case where a plurality of gain regions have the same carrier density is illustrated as an example, an effect may be obtained in a certain range even if the same carrier density is not employed.

Although the case of the carrier density which attains a gain of zero in the incident light wavelength in the non-gain region is illustrated, an effect may be obtained if the gain in the incident light wavelength is zero or less and driving may be performed by the gain of zero or driving may be performed by inverse bias.

Furthermore, in a case where a carrier density of the non-gain region is zero, a configuration in which the non-gain region and a driving system may not be connected to each other if the connection is not required or a configuration in which electrodes are not formed in the non-gain region may be employed.

Although the case where the incident light wavelengths of 1010 nm, 1040 nm, and 1080 nm attain the optimum driving state has been illustrated, the wavelength is not limited to these as long as the wavelength is included in a target wavelength range. However, it is preferable that incident light wavelengths disperse within the target wavelength range.

Although the case where the three-electrode structure is illustrated, an effect of the present disclosure is obtained when at least two electrodes are used (refer to a second embodiment as for the two-electrode structure).

A large number of short electrodes (10 μm, for example) may be disposed. By this, an obtained gain length becomes close to a gain length which attains the optimum driving state (refer to a third embodiment as for an electrode structure for finely setting a gain region). With this configuration, however, absorption in the non-electrode region may be increased, and therefore, control of the electrode structure and control of a driving state may be difficult.

Although the case where L₁ is 417 μm, L₂ is 504 μm, and L₃ is 2709 μm is illustrated as an example in this embodiment, the same effect may be obtained if a length of the electrode and carrier density of the electrode are coupled and are replaced with another couple in a device.

Although the case where L₁ is 417 μm, L₂ is 504 μm, and L₃ is 2709 μm is illustrated as an example in this embodiment, the same effect may be obtained if L₁ is 417 μm, L₂ is 920 μm, and L₃ is 2293 82 m.

Although the three-electrode structure is illustrated, the same effect may be obtained if the number of electrodes is larger in a substantially the same driving state. For example, the driving state in the incident light wavelength of 1010 nm (L₁=417 [μm], L₂=504 [μm], L₃=2709 [μm], N₁=8.0E+18 [/cm³], and N₂=N₃=3.0E+18 [/cm³]) illustrated in FIG. 11 may be changed. For example, even if the driving state is changed to a driving state (L₁=200 [μm], L₂=217 [μm], L₃=504 [μm], L₄=2709 [μm], N₁=N₂=8.0E+18 [/cm³], and N₃=N₄=3.0E+18 [/cm³], the same driving states are seen to be substantially the same.

Lₐ may be designed to be long as long as the driving state is not influenced. However, if Lₐ is long, an amount of unnecessary ASE light is increased, Lₐ is preferably as short as possible.

### Method for Controlling Gain Spectrum of SOA

Another configuration example of the SOA and the wavelength sweeping light source according to the foregoing embodiment will be described.

In this configuration example, first, the wavelength sweeping light source 102 is driven and emitted light is divided by a beam splitter (not illustrated). A portion of the divided light is detected by a line sensor (not illustrated) as monitor light and a signal corresponding to a center wavelength of the monitor light is transmitted to the controller 105. Then current is supplied to the electrodes of the SOA 104 based on the signal. With this configuration, the SOA 104 may be controlled to have a gain spectrum corresponding to a wavelength of light actually emitted from the wavelength sweeping light source.

Furthermore, the relationship between a voltage value and an oscillation wavelength for controlling displacement of the MEMS mirror of the wavelength sweeping light source 102 may be recognized in advance. Specifically, the function generator 101 may transmit the same signal to a voltage amplifier (not illustrated) which controls MEMS driving of the wavelength sweeping light source 102 and the current controller 105 of the SOA so that the SOA has a gain spectrum corresponding to light emitted from the wavelength sweeping light source.

Furthermore, a memory (not illustrated) which stores a table including the correspondence relationship between a temporal change of a wavelength of light emitted from the wavelength sweeping light source 102 and a current value required to be supplied to the SOA so as to perform optical amplification suitable for each wavelength of emitted light may be included in the structure.

### Optical Amplification Method

An optical amplification method for amplifying incident light using the optical amplifier according to the foregoing embodiment will be described. The optical amplification method according to this embodiment uses a semiconductor optical amplifier as described in the first embodiment. Specifically, one of electrode layers included in the semiconductor optical amplifier includes an electrode group having at least two electrodes which are separated from each other in a waveguide direction of the waveguide of the light emitted from the semiconductor optical amplifier.

The optical amplification method according to this embodiment at least has three steps below.
(1) A step of causing light to enter the semiconductor optical amplifier.
(2) A step of amplifying intensity of light incident on the semiconductor optical amplifier.
(3) A step of emitting light having the intensity amplified in the amplification step from the semiconductor optical amplifier.

The amplification step (3) includes a step of changing an optical amplification factor in accordance with a wavelength of the incident light by independently supplying current to different regions in the active layer of the semiconductor optical amplifier using the at least two electrodes of the semiconductor optical amplifier.

Furthermore, a region in the waveguide which attains a positive gain in the active layer in a wavelength of incident light is defined as a gain region, and a total length of gain regions along the waveguide is defined as a gain length. In this case, the amplification step preferably includes a step of changing the gain length in accordance with the wavelength of the incident light.

Furthermore, the amplification step preferably includes a step of reducing the gain length as the wavelength of the incident light becomes shorter.

Moreover, the amplification step includes a step of supplying current to the active layer so that the carrier density in the active layer becomes larger as the wavelength of the incident light becomes shorter.

Furthermore, the amplification step includes a step of reducing the gain region as the wavelength of the incident light becomes shorter.

### Second Embodiment

A second embodiment of the present invention will be described hereinafter.

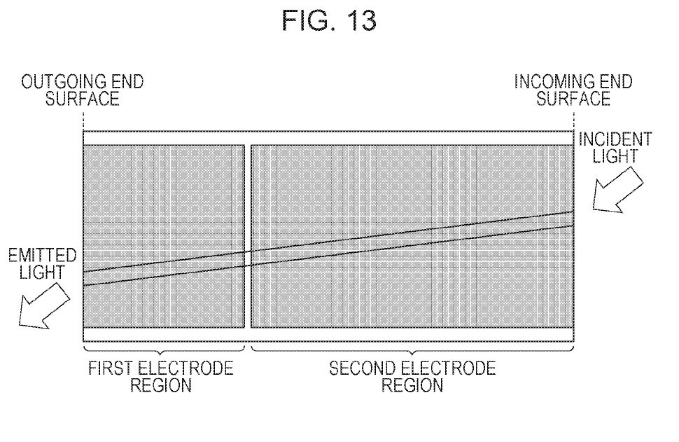

A configuration of an element of an SOA according to this embodiment will be described with reference to FIG. 13. This embodiment is the same as the first embodiment except for an electrode structure and a driving state. Therefore, only a difference from the first embodiment will be described.

This embodiment is characterized in that an upper electrode layer includes an electrode group having two electrodes. Accordingly, derivation of an optimum driving state and actual driving may be easily performed.

In this embodiment, design of lengths of the electrodes in the optimum driving state for incident light wavelengths of 1020 nm and 1070 nm is considered in this embodiment.

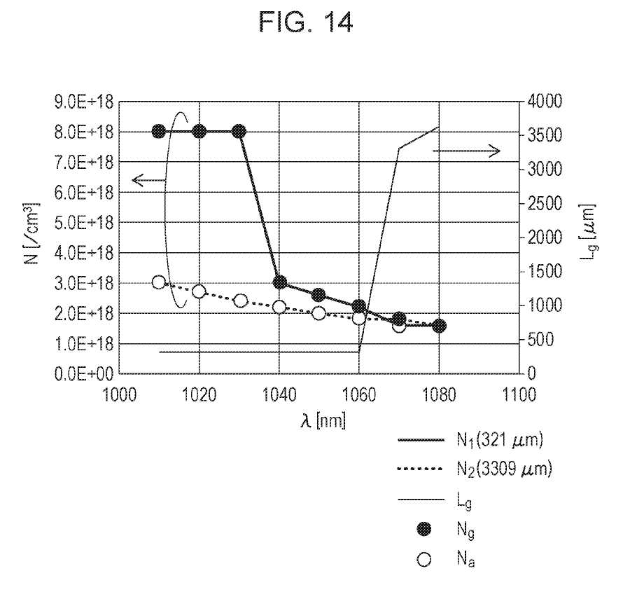

The optimum driving state is derived by the method of the first embodiment, and N₁, N₂, and L_{g} which attain the optimum driving state for an incident light wavelength λ are summarized as illustrated in FIG. 14.

Note that plot points of N_{g} are denoted by black circles and plot points of Nₐ are denoted by white circles. Note that an electrode structure in FIG. 14 (L₁=321 [μm] and L₂=3309 [μm]) is merely an example, and the same effect may be obtained even by switching lengths of electrodes and driving states of the electrodes.

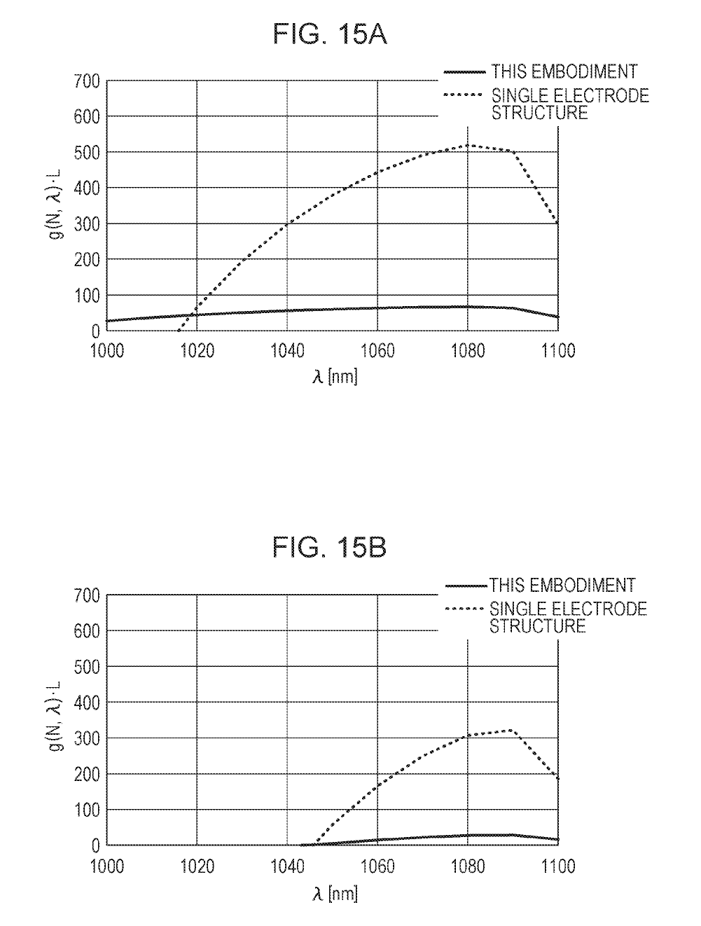

When g(N, λ)·L which attains the optimum driving states in the incident light wavelengths of 1030 nm and 1060 nm in the SOA of this embodiment and those in an SOA having a single electrode structure are compared with each other, results in FIGS. 15A and 15B are obtained, respectively. Accordingly, according to the comparison in the same incident light wavelength, an amount of ASE light generated in the SOA according to this embodiment may be more considerably reduced than that in the SOA having the single electrode structure.

### Third Embodiment

A third embodiment of the present invention will be described hereinafter.

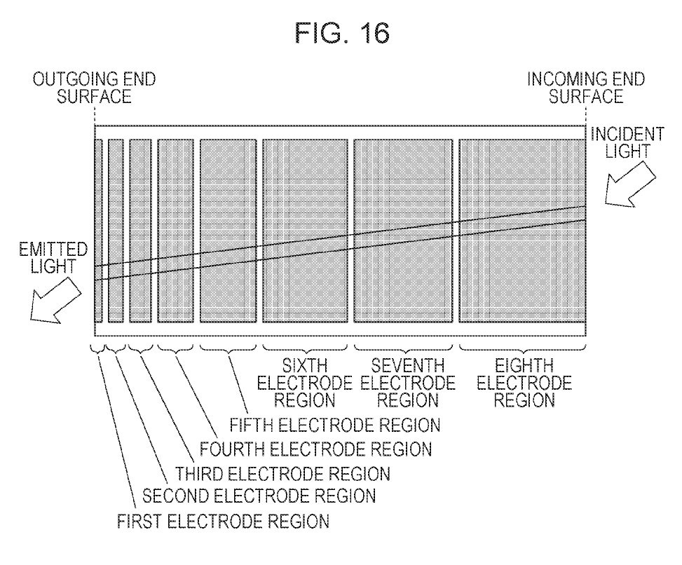

A configuration of an element of the SOA according to this embodiment will be described with reference to FIG. 16. This embodiment is the same as the first embodiment except for an electrode structure and a driving state. Therefore, only a difference from the first embodiment will be described.

This embodiment is characterized in an electrode structure in which lengths of electrodes are regularly changed. In this way, a combination of a gain length and a non-gain length may be adjusted with a higher degree of freedom when compared with the first embodiment.

In the first embodiment, lengths of the electrodes are set so that the gain length which attains the optimum driving state for a certain incident light wavelength are obtained. However, with this configuration, gain lengths which attain optimum driving states may not be obtained in other wavelengths. According to this embodiment, the lengths of the electrodes are designed so that combinations of the lengths of the electrodes which attains lengths closer to the gain lengths which attain the optimum driving states for all incident light wavelengths in a target wavelength range are obtained. For example, electrodes which satisfy “Lₖ=2^{m}·L₁ (k, l, m: natural numbers)” are obtained as much as possible. Specifically, as illustrated in FIG. 16, L₁ to L₈ correspond to 20 μm, 40 μm, 80 μm, 160 μm, 320 μm, 640 μm, 1280 μm, and 1090 μm, (in random order). By this, a gain length which attains an optimum driving state may be obtained in the incident light wavelength 1080 nm corresponding to the largest gain length which attains the optimum driving state. In addition, by a combination of lengths of electrodes, differences between the gain lengths which attain the optimum driving states in the individual incident light wavelengths and actual gain lengths may be suppressed to be less than 10 μm.

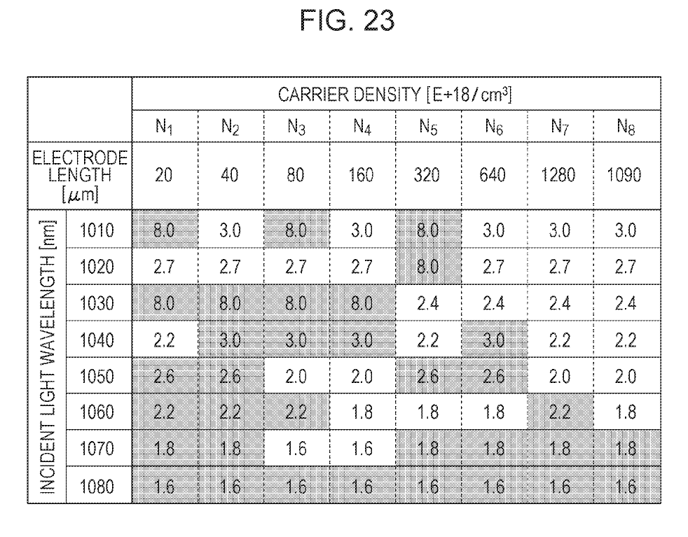

The optimum driving states are derived by the method described in the first embodiment, and lengths of electrode regions which attain the optimum driving states for the incident light wavelengths and carrier densities are summarized as illustrated in FIG. 23. However, the illustrated electrode structure (L₁=20 [μm], L₂=40 [μm], L₃=80 [μm], L₄=160 [μm], L₅=320 [μm], L₆=640 [μm], L₇=1280 [μm], and L₈=1090 [μm]) is merely an example. The same effect may be attained even if the lengths of the electrodes and driving states corresponding to the electrodes are switched.

Note that colored carrier densities represent carrier densities in a non-gain region.

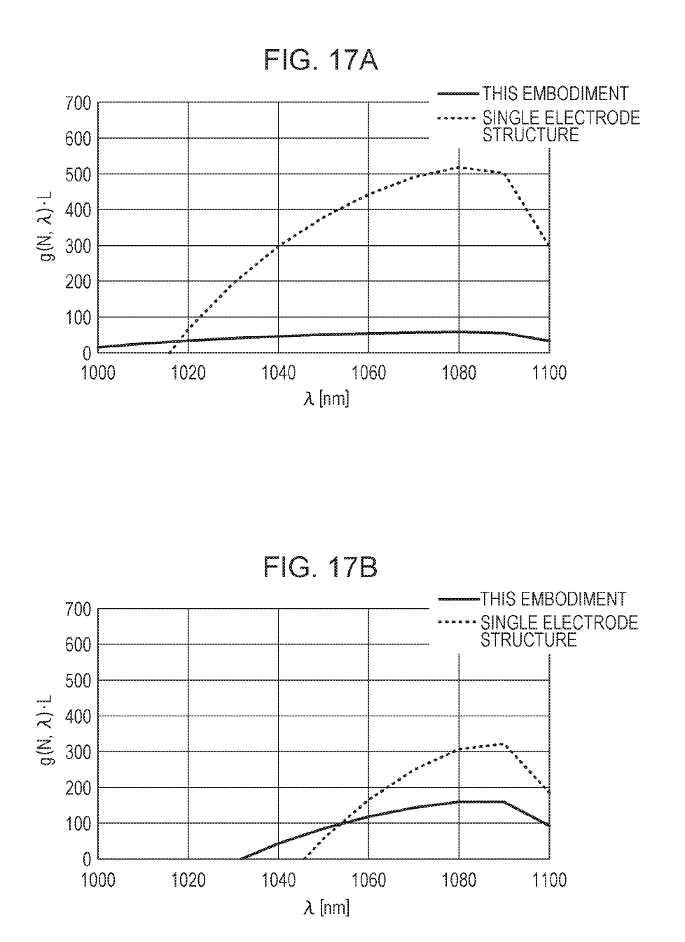

When g (N, λ)·L which attains the optimum driving states in the incident light wavelengths of 1030 nm and 1060 nm in the SOA of this embodiment and those in an SOA having a single electrode structure are compared with each other, results in FIGS. 17A and 17B are obtained, respectively. According to the comparison in the same incident light wavelength, an amount of ASE light generated in the SOA according to this embodiment may be more considerably reduced than that in the SOA having the single electrode structure.

Although the case where a minimum unit of an electrode is 20 μm is described as an example in this embodiment, the same effect as this embodiment may be obtained in a case of values of 10 μm, 50 μm, or 100 μm. However, if the minimum unit is less than 10 μm, absorption in the non-electrode region is increased, and it may be difficult to control the electrode structure and the driving state.

Although the case where the number of combinations of lengths of electrodes which satisfy “Lₖ=2^{m}·L₁” is 7 is described as an example, the same effect as this embodiment may be obtained as long as the number of combinations is 1 or more.

### Fourth Embodiment

A fourth embodiment of the present invention will be described hereinafter.

This embodiment is the same as the first embodiment except for target emitted light. Therefore, only a difference from the first embodiment will be described. Although a sample structure is as illustrated in FIGS. 3A to 3C, lengths of electrodes and driving states are different from the first embodiment.

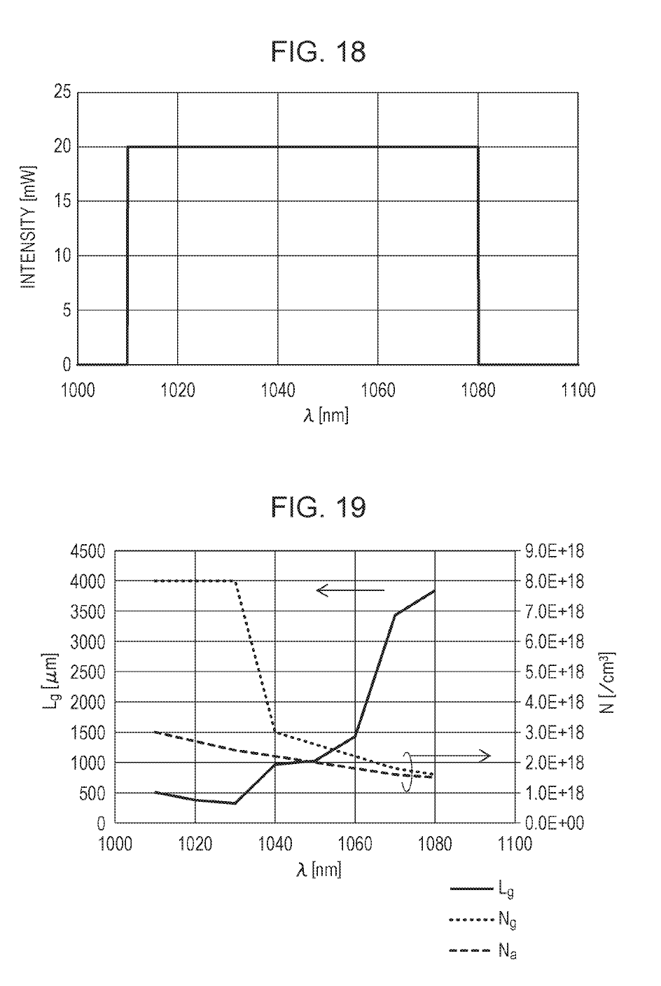

It is assumed that target emitted light in this embodiment has a sweeping spectrum of a rectangle form having a light intensity of 20 mW in a wavelength range from 1010 nm to 1080 nm (FIG. 18).

A method for deriving an optimum driving state is described in the first embodiment.

In this embodiment, design of lengths of the electrodes in optimum driving states for incident light wavelengths of 1010 nm, 1040 nm, and 1080 nm, for example, is considered.

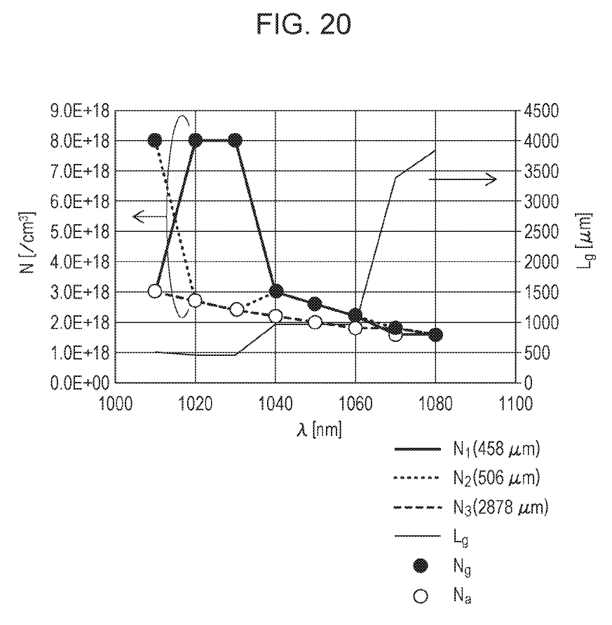

Results of obtainment of L_{g}, N_{g}, and Nₐ which attain the optimum driving states for the target wavelength range from 1010 nm to 1080 nm are illustrated in FIG. 19. Furthermore, the optimum driving states are derived and N₁, N₂, N₃, and L_{g} which attain the optimum driving state for the incident light wavelength λ are summarized as illustrated in FIG. 20.

Note that plot points of N_{g} are denoted by black circles and plot points of Nₐ are denoted by white circles. Note that an electrode structure here (L₁=458 [μm], L₂=506 [μm], and L₃=2878 [μm]) is merely an example, and the same effect may be obtained even by switching lengths of electrodes and driving states of the electrodes.

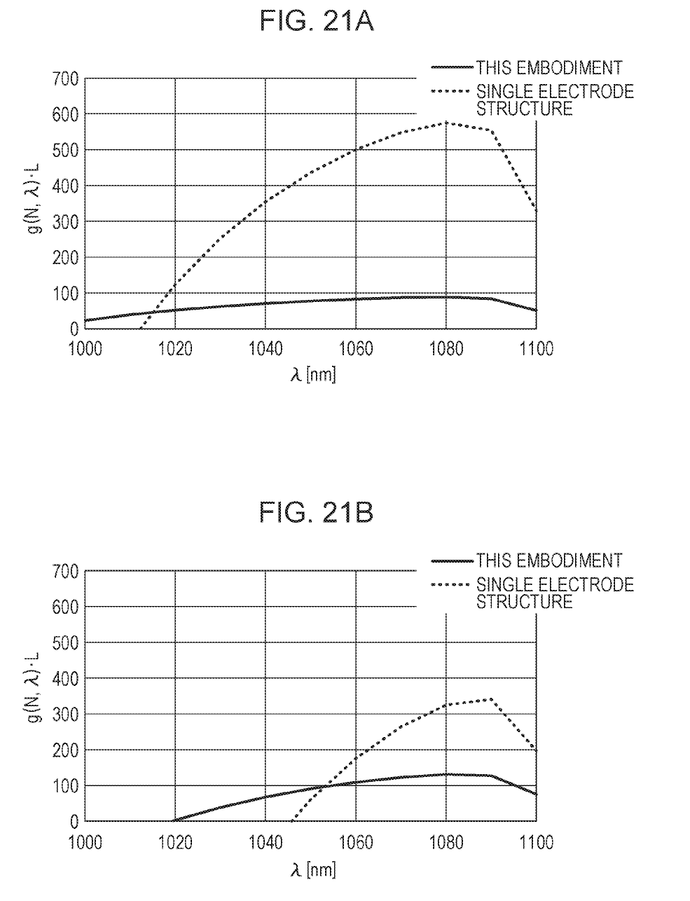

When g(N, λ)·L which attains the optimum driving states in the incident light wavelengths of 1030 nm and 1060 nm in the SOA of this embodiment and those in an SOA having a single electrode structure are compared with each other, results in FIGS. 21A and 21B are obtained, respectively. According to the comparison in the same incident light wavelength, an amount of ASE light generated in the SOA according to this embodiment is smaller than that in the SOA having the single electrode structure.

### Fifth Embodiment

A fifth embodiment of the present invention will be described hereinafter.

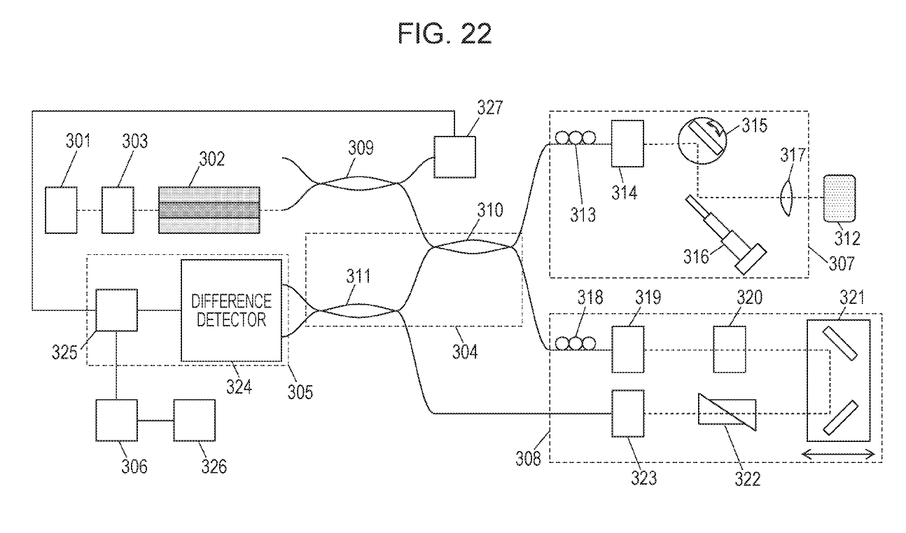

A configuration of this embodiment will be described with reference to FIG. 22. In this embodiment, an example of an OCT apparatus using the SOA of the present invention will be described.

The OCT apparatus includes a light source unit 301 (MEMS-VCSEL) which sweeps a frequency of emitted light, an optical amplifier (SOA) 302 which increases optical output and which control a sweeping spectrum form, and an isolator 303 disposed between the light source unit 301 and the optical amplifier (SOA) 302. The OCT apparatus further includes an interfering unit 304 which generates interfering light, a signal output unit 305 which receives the interfering light and outputs an interfering signal, a signal obtaining unit 306 which obtains information on an object (subject) based on the interfering signal. Furthermore, the OCT apparatus includes a measurement arm (an irradiation optical system) 307 and a reference arm (a reference optical system) 308.

The interfering unit 304 includes two couplers 310 and 311. First, the coupler 310 divides light emitted from a light source into irradiation light to be emitted to a subject 312 and reference light. The irradiation light is incident on the subject 312 through the measurement arm 307. Specifically, a polarization state of the irradiation light which is incident on the measurement arm 307 is formed by a polarization controller 313 before the irradiation light is emitted from a collimator 314 as spatial light. Thereafter, the irradiation light is incident on the subject 312 through an X-axis scanner 315, a Y-axis scanner 316, and a focus lens 317.

Note that the X-axis scanner 315 and the Y-axis scanner 316 are a scanning unit having a function of scanning the subject 312 with the irradiation light. An irradiation position of the irradiation light on the subject 312 may be changed by the scanning unit. Then backscattered light (reflection light) from the subject 312 is emitted from the measurement arm 307 again through the focus lens 317, the Y-axis scanner 316, the X-axis scanner 315, the collimator 314, and the polarization controller 313. Then the backscattered light is incident on the coupler 311 through the coupler 310.

Note that the interfering unit 304, the measurement arm 307, and the reference arm 308 may be collectively referred to as an interference optical system. Although the interference optical system in FIG. 22 is a Mach-Zehnder type, a Michelson type may be used.

On the other hand, the reference light is incident on the coupler 311 through the reference arm 308. Specifically, a polarization state of the reference light which is incident on the reference arm 308 is formed by a polarization controller 318 before the reference light is emitted from a collimator 319 as spatial light. Thereafter, the reference light is incident on an optical fiber through a dispersion compensation glass 320, an optical path adjustment optical system 321, a dispersion control prism pair 322, and a collimator lens 323, and further emitted from the reference arm 308 and incident on the coupler 311.

The reflection light of the subject 312 which has passed the measurement arm 307 and the light which has passed the reference arm 308 interfere with each other in the coupler 311. The interfering light is detected by the signal output unit 305. The signal output unit 305 includes a difference detector 324 and an analog/digital (A/D) converter 325. First, in the signal output unit 305, the difference detector 324 detects the interfering light divided immediately after generation of the interfering light in the coupler 311. Then the A/D converter 325 converts an interfering signal which has been converted into an electric signal by the difference detector 324 into a digital signal. The digital signal is transmitted to the signal obtaining unit 306 and subjected to frequency analysis, such as Fourier transform, so that information on the subject 312 is obtained. The obtained information on the subject 312 is displayed as a tomographic image by a display unit 326.

The OCT apparatus in FIG. 22 performs sampling of the interfering light at an equal optical frequency interval (an equal wavelength interval) based on a k clock signal output from a k-clock generation unit 327 disposed outside the light source.

Furthermore, a coupler 309 is disposed so as to branch a portion of the light emitted from the light source into the k-clock generation unit 327.

Note that the k-clock generation unit 327 and the coupler 309 may be incorporated in the light source unit 301 or the SOA 302.

The process of obtaining information on tomography at a certain point of the subject 312 has been described above and such a process of obtaining information on tomography in a depth direction of the subject 312 is referred to as “A-scan”.

Furthermore, information on the tomography of the subject 312 in a direction orthogonal to A-scan, that is, a scanning direction for obtaining a two-dimensional image is referred to as “B-scan”, and a process of performing scanning in a direction orthogonal to the scanning directions of A-scan and B-scan is referred to as “C-scan”. Specifically, in a case where a two-dimensional raster scanning is performed on an ocular fundus plane so that a three-dimensional tomographic image is obtained, a direction of high-speed scanning is referred to as “B-scan” and a direction of low-speed scanning which is orthogonal to B-scan is referred to as “C-scan”. A two-dimensional tomographic image may be obtained by performing A-scan and B-scan and a three-dimensional tomographic image may be obtained by performing A-scan, B-scan, and C-scan. B-scan and C-scan are performed by the X-axis scanner 315 and the Y-axis scanner 316 described above.

Note that the X-axis scanner 315 and the Y-axis scanner 316 are configured by respective deflection mirrors having rotation shafts disposed so as to be orthogonal to each other. The X-axis scanner 315 performs scanning in an X-axis direction and the Y-axis scanner 316 performs scanning in a Y-axis direction. The X-axis direction and the Y-axis direction are orthogonal to a normal of a surface of the subject and orthogonal to each other.

Furthermore, the line scanning direction of B-scan and C-scan may not coincide with the X-axis direction or the Y-axis direction. Therefore, the line scanning directions of B-scan and C-scan may be appropriately determined in accordance with a two-dimensional tomographic image or a three-dimensional tomographic image.

This embodiment is characterized by the SOA, and when the SOAs of the present invention disclosed as the foregoing embodiments are used, ASE light may be reduced while a sweeping spectrum form of MEMS-VCSEL is controlled, and accordingly, information on a high-resolution tomographic image may be advantageously obtained. The OCT apparatus is mainly useful in tomographic image shooting in ophthalmology.

The present invention is not limited to the foregoing embodiments, and various changes and modifications may be made without departing from the spirit and the scope of the present invention. Accordingly, the following claims are attached to disclose the scope of the present invention.

According to the optical amplifier of the present invention, at least one of electrode layers of a laminated body which constitutes the optical amplifier is divided into a plurality of portions so that a region subjected to amplification may be changed in addition to an amplification factor of the optical amplifier. Therefore, an amount of the ASE light including light having unrequired wavelengths may be reduced while sufficient light output intensity is obtained in a required wavelength.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An optical amplifier comprising:
a laminated body including two electrode layers and an active layer disposed between the electrode layers,
the laminated body including a waveguide which guides light in an in-plane direction of the active layer,
wherein the light which is incident on the laminated body is amplified and emitted from an end surface in the in-plane direction of the laminated body through the waveguide,
wherein at least one of the two electrode layers has an electrode group including at least two electrodes which are disposed separately from each other in a waveguide direction of the waveguide, and
wherein an amplification factor of the incident light is changeable in accordance with a wavelength of the incident light by independently supplying current to different regions in the active layer using the at least two electrodes.

2. The optical amplifier according to claim 1, further comprising a controller configured to independently control current to be supplied to the different regions in the active layer using the at least two electrodes.

3. The optical amplifier according to claim 1, wherein, when a region in the waveguide in which a positive gain of the active layer is obtained in the wavelength of the incident light is defined as a gain region and a total length of gain regions along the waveguide is defined as a gain length, the gain length is changeable in accordance with a wavelength of the incident light.

4. The optical amplifier according to claim 1, wherein the gain length is reduced as the wavelength of the incident light becomes shorter.

5. The optical amplifier according to claim 1, wherein density of current to be supplied to the active layer is increased as the wavelength of the incident light is shorter.

6. The optical amplifier according to claim 1, wherein the number of electrodes, in the electrode group, to be used for supplying the current to the active layer is reduced as the wavelength of the incident light becomes shorter.

7. The optical amplifier according to claim 1, wherein a waveform in temporal change of a wavelength of light emitted from the optical amplifier has a substantially Gaussian form, a substantially rectangle form, or a substantially cosine taper form.

8. The optical amplifier according to claim 1, wherein the active layer has an asymmetry quantum well structure.

9. A light source system comprising:
a light source unit configured to change a wavelength of light to be emitted; and
the optical amplifier according to claim 1 which amplifies light emitted from the light source unit.

10. The light source system according to claim 9, wherein the light source unit is a surface emission laser.

11. An optical coherence tomography, comprising:
a light source unit configured to change a wavelength of light to be emitted;
the optical amplifier according to claim 1 which amplifies the light emitted from the light source unit;
an interference optical system configured to divide light emitted from the optical amplifier into irradiation light which is incident on an object through an irradiation optical system and reference light which passes a reference optical system and configured to generate interfering light generated by reflection light of the light which is incident on the object and the reference light;
a signal output unit configured to receive the interfering light and output an interfering signal; and
an obtaining unit configured to obtain information on the object based on the interfering signal.

12. The optical coherence tomography according to claim 11 having the light source unit which is a surface emission laser.

13. An optical amplification method for amplifying incident light using a semiconductor optical amplifier,
the semiconductor optical amplifier including electrode layers, at least one of which includes an electrode group having at least two electrodes which are separated from each other in a waveguide direction of an optical waveguide of the semiconductor optical amplifier,
the optical amplification method, comprising:
emitting light to the semiconductor optical amplifier;
amplifying intensity of light incident on the semiconductor optical amplifier; and
emitting light having the amplified intensity from the semiconductor optical amplifier;
wherein the amplifying includes a change of an optical amplification factor in accordance with a wavelength of the incident light by independently supplying current to different regions in an active layer of the semiconductor optical amplifier using the at least two electrodes.

14. The optical amplification method according to claim 13, wherein, when a region in the waveguide in which a positive gain in the active layer in a wavelength of incident light is obtained is defined as a gain region, and a total length of gain regions along the waveguide is defined as a gain length, the amplifying includes a change of the gain length in accordance with the wavelength of the incident light.

15. The optical amplification method according to claim 13, wherein the amplifying includes reduction of the gain length as the wavelength of the incident light is shorter.

16. The optical amplification method according to claim 13, wherein the amplifying includes supply of current to the active layer so that carrier density in the active layer is increased as the wavelength of the incident light is shorter.

17. The optical amplification method according to claim 13, wherein the amplifying includes reduction of the gain region as the wavelength of the incident light is shorter.
